(19) United States
(12) Patent Application Publication
(10) Pub. No.: US 2017/0022260 A9
(43) Pub. Date: Jan. 26, 2017

(54) EXENDIN-4 PEPTIDE ANALOGUES AS DUAL GLP-1/GLUCAGON RECEPTOR AGONISTS

(71) Applicant: Sanofi, Paris (FR)

(72) Inventors: Torsten Haack, Frankfurt am Main (DE); Michael Wagner, Frankfurt am Main (DE); Bernd Henkel, Frankfurt am Main (DE); Siegfried Stengelin, Frankfurt am Main (DE); Andreas Evers, Frankfurt am Main (DE); Martin Bossart, Frankfurt am Main (DE); Dieter Kadereit, Frankfurt am Main (DE)

(21) Appl. No.: 14/569,326

(22) Filed: Dec. 12, 2014

(30) Foreign Application Priority Data
Dec. 13, 2013 (EP) 13306715.7

(51) Int. Cl. C07K 14/605; A61K 49/14; A61K 49/04; A61K 38/26; A61K 45/06

(52) U.S. Cl. CPC C07K 14/605; A61K 38/26; A61K 45/06; A61K 49/04; A61K 49/14

(57) ABSTRACT

The present invention relates to exendin-4 derivatives and their medical use, for example in the treatment of disorders of the metabolic syndrome, including diabetes and obesity, as well as reduction of excess food intake

### FIELD OF THE INVENTION

The present invention relates to exendin-4 peptide derivatives which—in contrast to the pure GLP-1 agonist exendin-4—activate both the GLP-1 and the Glucagon receptor and their medical use, for example in the treatment of disorders of the metabolic syndrome, including diabetes and obesity, as well as for reduction of excess food intake.

### BACKGROUND OF THE INVENTION

Exendin-4 is a 39 amino acid peptide which is produced by the salivary glands of the Gila monster (Heloderma suspectum) (Eng, J. et al., J. Biol. Chem., 267:7402-05, 1992). Exendin-4 is an activator of the glucagon-like peptide-1 (GLP-1) receptor, whereas it does not activate significantly the glucagon receptor.

Exendin-4 shares many of the glucoregulatory actions observed with GLP-1. Clinical and nonclinical studies have shown that exendin-4 has several beneficial antidiabetic properties including a glucose dependent enhancement in insulin synthesis and secretion, glucose dependent suppression of glucagon secretion, slowing down gastric emptying, reduction of food intake and body weight, and an increase in beta-cell mass and markers of beta cell function (Gentilella R et al., Diabetes Obes Metab., 11:544-56, 2009; Norris S L et al, Diabet Med., 26:837-46, 2009; Bunck M C et al, Diabetes Care., 34:2041-7, 2011).

These effects are beneficial not only for diabetics but also for patients suffering from obesity. Patients with obesity have a higher risk of getting diabetes, hypertension, hyperlipidemia, cardiovascular and musculoskeletal diseases.

Relative to GLP-1, exendin-4 is resistant to cleavage by dipeptidyl peptidase-4 (DPP4) resulting in a longer half-life and duration of action in vivo (Eng J., Diabetes, 45 (Suppl 2):152A (abstract 554), 1996).

Exendin-4 was also shown to be much more stable towards degradation by neutral endopeptidase (NEP), when compared to GLP-1, glucagon or oxyntomodulin (Endocrinology, 150(4), 1712-1721, 2009). Nevertheless, exendin-4 is chemically labile due to methionine oxdiation in position 14 (Hargrove D M et al., Regul. Pept., 141: 113-9, 2007) as well as deamidation and isomerization of asparagine in position 28 (WO 2004/035623).

The amino acid sequence of exendin-4 is shown as SEQ ID NO: 1

HGEGTFTSDLSKQMEEEAVRLFIEWLKNGGPSSGAPPPS-NH₂

The amino acid sequence of GLP-1(7-36)-amide is shown as SEQ ID NO 2

HAEGTFTSDVSSYLEGQAAKEFIAWLVKGR-NH₂

Liraglutide is a marketed chemically modified GLP-1 analog in which, among other modifications, a fatty acid is linked to a lysine in position 20 leading to a prolonged duration of action (Drucker D J et al, Nature Drug Disc. Rev. 9, 267-268, 2010; Buse, J. B. et al., Lancet, 374:39-47, 2009).

The amino acid sequence of Liraglutide is shown as SEQ ID NO 4.

HAEGTFTSDVSSYLEGQAAK((S)-4-Carboxy-4-hexa-
decanoylamino-butyryl-)EFIAWLVRGRG-OH

Glucagon is a 29-amino acid peptide which is released into the bloodstream when circulating glucose is low. Glucagon's amino acid sequence is shown in SEQ ID NO 3.

HSQGTFTSDYSKYLDSRRAQDFVQWLMNT-OH

During hypoglycemia, when blood glucose levels drop below normal, glucagon signals the liver to break down glycogen and release glucose, causing an increase of blood glucose levels to reach a normal level. Hypoglycemia is a common side effect of insulin treated patients with hyperglycemia (elevated blood glucose levels) due to diabetes. Thus, glucagon's most predominant role in glucose regulation is to counteract insulin action and maintain blood glucose levels.

Hoist (Hoist, J. J. Physiol. Rev. 2007, 87, 1409) and Meier (Meier, J. J. Nat. Rev. Endocrinol. 2012, 8, 728) describe that GLP-1 receptor agonists, such as GLP-1, liraglutide and exendin-4, have 3 major pharmacological activities to improve glycemic control in patients with T2DM by reducing fasting and postprandial glucose (FPG and PPG): (i) increased glucose-dependent insulin secretion (improved first- and second-phase), (ii) glucagon suppressing activity under hyperglycemic conditions, (iii) delay of gastric emptying rate resulting in retarded absorption of meal-derived glucose.

Pocai et al (Obesity. 2012; 20:1566-1571; Diabetes 2009, 58, 2258) and Day et al. (Nat Chem Biol 2009; 5:749) describe that dual activation of the GLP-1 and glucagon receptors, e.g., by combining the actions of GLP-1 and glucagon in one molecule leads to a therapeutic principle with anti-diabetic action and a pronounced weight lowering effect

Peptides which bind and activate both the glucagon and the GLP-1 receptor (Hjort et al. Journal of Biological Chemistry, 269, 30121-30124, 1994; Day J W et al, Nature Chem Biol, 5: 749-757, 2009) and suppress body weight gain and reduce food intake are described in patent applications WO 2008/071972, WO 2008/101017, WO 2009/155258, WO 2010/096052, WO 2010/096142, WO 2011/075393, WO 2008/152403, WO 2010/070251, WO 2010/070252, WO 2010/070253, WO 2010/070255, WO 2011/160630, WO 2011/006497, WO 2011/152181, WO 2011/152182, WO2011/117415, WO2011/117416, and WO 2006/134340, the contents of which are herein incorporated by reference.

In addition, triple co-agonist peptides which not only activate the GLP-1 and the glucagon receptor, but also the GIP receptor are described in WO 2012/088116 and by V A Gault et al (Biochem Pharmacol, 85, 16655-16662, 2013; Diabetologia, 56, 1417-1424, 2013).

Bloom et al. (WO 2006/134340) disclose that peptides which bind and activate both the glucagon and the GLP-1 receptor can be constructed as hybrid molecules from glucagon and exendin-4, where the N-terminal part (e.g. residues 1-14 or 1-24) originate from glucagon and the C-terminal part (e.g. residues 15-39 or 25-39) originate from exendin-4.

D E Otzen et al (Biochemistry, 45, 14503-14512, 2006) disclose that N- and C-terminal hydrophobic patches are involved in fibrillation of glucagon, due to the hydrophobicity and/or high β-sheet propensity of the underlying residues.

Compounds of this invention are exendin-4 peptide analogues comprising leucine in position 10 and glutamine in position 13.

Krstenansky et al (Biochemistry, 25, 3833-3839, 1986) show the importance of the residues 10-13 of glucagon (YSKY) for its receptor interactions and activation of adenylate cyclase. In the exendin-4 derivatives described in this invention, several of the underlying residues are different from glucagon. In particular residues Tyr10 and Tyr13, which are known to contribute to the fibrillation of glucagon (D E Otzen, Biochemistry, 45, 14503-14512, 2006) are replaced by Leu in position 10 and Gln, a non-aromatic polar amino acid, in position 13. This replacement, especially in combination with isoleucine in position 23 and glutamate in position 24 leads to exendin-4 derivatives with potentially improved biophysical properties as solubility or aggregation behavior in solution. The non-conservative replacement of an aromatic amino acid with a polar amino acid in position 13 of an exendin-4 analogue surprisingly leads to peptides with high activity on the glucagon receptor and optionally on the GIP receptor.

Compounds of this invention are more resistant to cleavage by neutral endopeptidase (NEP) and dipeptidyl peptidase-4 (DPP4), resulting in a longer half-life and duration of action in vivo, when compared with GLP-1 and glucagon.

Compounds of this invention preferably are soluble not only at neutral pH, but also at pH 4.5. This property potentially allows co-formulation for a combination therapy with an insulin or insulin derivative and preferably with a basal insulin like insulin glargine/Lantus®.

### BRIEF SUMMARY OF THE INVENTION

Provided herein are exendin-4 derivatives which potently activate the GLP1 and the glucagon receptor and optionally the GIP receptor. In these exendin-4 derivatives—among other substitutions—methionine at position 14 is replaced by leucin

The invention provides a peptidic compound having the formula (I):

R¹—Z—R² (I)

wherein Z is a peptide moiety having the formula (II)

(II)
His-X2-X3-Gly-Thr-Phe-Thr-Ser-Asp-Leu-Ser-Lys-Gln-
Leu-Asp-Glu-Gln-X18-Ala-X20-X21-Phe-Ile-Glu-Trp-
Leu-Ile-X28-Gly-Gly-Pro-X32-Ser-Gly-Ala-Pro-Pro-
Pro-Ser

X2 represents an amino acid residue selected from Ser, D-Ser and Aib,
X3 represents an amino acid residue selected from Gln and His,
X18 represents an amino acid residue selected from Arg and Lys
X20 represents an amino acid residue selected from Lys, Gln, His and (S)MeLys,
X21 represents an amino acid residue selected from Asp and Glu,
X28 represents an amino acid residue selected from Ser and Ala,
X32 represents an amino acid residue selected from Ser and Val,
R¹ represents NH₂,
R² represents OH or NH₂,
or a salt or solvate thereof.

The compounds of the invention are GLP-1 and glucagon receptor agonists and optionally GIP receptor agonists as determined by the observation that they are capable of stimulating intracellular cAMP formation.

According to another embodiment the peptidic compounds of the invention exhibit a relative activity of at least 0.1% (i.e. EC50<700 pM), preferably at least 0.7% (i.e. EC50<100 pM), more preferably at least 1.4% (i.e. EC50<50 pM) and even more preferably at least 7% (i.e. EC50<10 pM) compared to that of GLP-1(7-36) at the GLP-1 receptor (EC50=0.7 pM).

According to another embodiment the peptidic compounds of the invention exhibit a relative activity of at least 0.1% (i.e. EC50<1000 pM), preferably of at least 0.33% (i.e. EC50<300 pM), more preferably at least 1% (i.e. EC50<100 pM) and even more preferably at least 1.43% (i.e. EC50<70 pM) compared to that of natural glucagon at the glucagon receptor (EC50=1.0 pM).

The term “activity” as used herein preferably refers to the capability of a compound to activate the human GLP-1 receptor and the human glucagon receptor. More preferably the term “activity” as used herein refers to the capability of a compound to stimulate intracellular cAMP formation. The term “relative activity” as used herein is understood to refer to the capability of a compound to activate a receptor in a certain ratio as compared to another receptor agonist or as compared to another receptor. The activation of the receptors by the agonists (e.g. by measuring the cAMP level) is determined as described herein, e.g. as described in the examples.

The compounds of the invention preferably have an EC₅₀ for hGLP-1 receptor of 100 pmol or less, more preferably of 90 pmol or less, more preferably of 80 pmol or less, more preferably of 70 pmol or less, more preferably of 60 pmol or less, more preferably of 50 pmol or less, more preferably of 40 pmol or less, more preferably of 30 pmol or less, more preferably of 25 pmol or less, more preferably of 20 pmol or less, more preferably of 15 pmol or less, more preferably of 10 pmol or less, more preferably of 9 pmol or less, more preferably of 8 pmol or less, more preferably of 7 pmol or less, more preferably of 6 pmol or less, and more preferably of 5 pmol or less more preferably of 4 pmol or less, more preferably of 3 pmol or less, and more preferably of 2 pmol or less and/or an EC₅₀ for hGlucagon receptor of 300 pmol or less, preferably of 200 pmol or less; more preferably of 150 pmol or less, more preferably of 100 pmol or less, more preferably of 90 pmol or less, more preferably of 80 pmol or less, more preferably of 70 pmol or less, more preferably of 60 pmol or less, more preferably of 50 pmol or less, more preferably of 40 pmol or less, more preferably of 30 pmol or less, more preferably of 25 pmol or less, more preferably of 20 pmol or less, more preferably of 15 pmol or less, more preferably of 10 pmol or less. It is particularly preferred that the EC₅₀ for both receptors is 100 pmol or less, more preferably of 90 pmol or less, more preferably of 80 pmol or less, more preferably of 70 pmol or less, more preferably of 60 pmol or less, more preferably of 50 pmol or less, more preferably of 40 pmol or less, more preferably of 30 pmol or less, more preferably of 25 pmol or less, more preferably of 20 pmol or less, more preferably of 15 pmol or less, more preferably of 10 pmol or less. The EC₅₀ for hGLP-1 receptor and hGlucagon receptor may be determined as described in the Methods herein and as used to generate the results described in Example 4.

According to another embodiment, the compounds of the invention have an EC₅₀ for hGIP receptor of 500 pM or less, more preferably 200 pM or less, more preferably 150 pM or less, more preferably 100 pM or less, more preferably 90 pM or less, more preferably 80 pM or less, more preferably 70 pM or less, more preferably 60 pM or less, more preferably 50 pM or less, more preferably 40 pM or less, more preferably 30 pM or less, more preferably 20 pM or less, more preferably of 10 pmol or less.

In still another embodiment, the EC₅₀ for all three receptors, i.e. for the hGLP1-receptor, for the hGlucagon receptor and for the hGIP receptor, is 500 pM or less, more preferably 200 pM or less, more preferably 150 pM or less, more preferably 100 pM or less, more preferably 90 pM or less, more preferably 80 pM or less, more preferably 70 pM or less, more preferably 60 pM or less, more preferably 50 pM or less, more preferably 40 pM or less, more preferably 30 pM or less, more preferably 20 pM or less, more preferably of 10 pmol or less.

The compounds of the invention have the ability to reduce the intestinal passage, increase the gastric content and/or to reduce the food intake of a patient. These activities of the compounds of the invention can be assessed in animal models known to the skilled person. Preferred compounds of the invention may increase the gastric content of mice, preferably of female NMRI-mice, if administered as a single subcutaneous dose, at least by 25%, more preferably by at least 30%, more preferably by at least 40%, more preferably by at least 50%, more preferably by at least 60%, more preferably by at least 70%, more preferably by at least 80%. Preferably, this result is measured 1 h after administration of the respective compound and 30 mins after administration of a bolus, and/or reduces intestinal passage of mice, preferably of female NMRI-mice, if administered as a single subcutaneous dose, at least by 45%; more preferably by at least 50%, more preferably by at least 55%, more preferably by at least 60%, and more preferably at least 65%; and/or reduces food intake of mice, preferably of female NMRI-mice, if administered as a single subcutaneous dose by at least 10%, more preferably 15%, and more preferably 20%.

The compounds of the invention have the ability to reduce blood glucose level, and/or to reduce HbA1c levels of a patient. These activities of the compounds of the invention can be assessed in animal models known to the skilled person and also described herein in the Methods. Preferred compounds of the invention may reduce blood glucose levels of mice, preferably in female leptin-receptor deficient diabetic db/db mice, if administered as a single subcutaneous dose of 0.1 mg/kg body weight by at least 4 mmol/L; more preferably by at least 8 mmol/L, more preferably by at least 12 mmol/L.

The compounds of the invention have the ability to reduce body weight of a patient. These activities of the compounds of the invention can be assessed in animal models known to the skilled person.

Surprisingly, it was found that peptidic compounds of the formula (I) showed very potent GLP-1 and Glucagon receptor activation

Furthermore, oxidation (in vitro or in vivo) of methionine, present in the core structure of exendin-4, is not possible anymore for peptidic compounds of the formula (I).

In one embodiment the compounds of the invention have a high solubility at acidic and/or physiological pH values, e.g., at pH 4.5 and/or at pH 7.4 at 25° C., in another embodiment at least 0.5 mg/ml and in a particular embodiment at least 1.0 mg/ml.

Furthermore, the compounds of the invention preferably have a high stability when stored in solution. Preferred assay conditions for determining the stability is storage for 7 days at 40° C. in solution at pH 4.5 or pH 7. The remaining amount of peptide is determined by chromatographic analyses as described in the Examples. Preferably, after 7 days at 40° C. in solution at pH 4.5 or pH 7 the remaining peptide amount is at least 80%, more preferably at least 85%, even more preferably at least 90% and even more preferably at least 95%.

Preferably, the compounds of the present invention comprise a peptide moiety Z (II) which is a linear sequence of 39 amino carboxylic acids, particularly α-amino carboxylic acids linked by peptide, i.e. carboxamide bonds.

A further embodiment relates to a group of compounds, wherein
R² is NH₂.

A further embodiment relates to a group of compounds, wherein
X2 represents an amino acid residue selected from Ser, D-Ser and Aib,
X3 represents an amino acid residue selected from Gln and His,
X18 represents Arg
X20 represents an amino acid residue selected from Lys, Gln, His and (S)MeLys,
X21 represents an amino acid residue selected from Asp and Glu,
X28 represents an amino acid residue selected from Ser and Ala,
X32 represents an amino acid residue selected from Ser and Val.

A further embodiment relates to a group of compounds, wherein
X2 represents Ser,
X3 represents an amino acid residue selected from Gln and His,
X18 represents Arg
X20 represents an amino acid residue selected from Lys and (S)MeLys,
X21 represents an amino acid residue selected from Asp and Glu,
X28 represents Ala,
X32 represents an amino acid residue selected from Ser and Val.

A further embodiment relates to a group of compounds, wherein
X2 represents D-Ser,
X3 represents an amino acid residue selected from Gln and His,
X18 represents Arg,
X20 represents an amino acid residue selected from Lys and (S)MeLys,
X21 represents an amino acid residue selected from Asp and Glu,
X28 represents Ala,
X32 represents an amino acid residue selected from Ser and Val.

A further embodiment relates to a group of compounds, wherein
X2 represents Aib,
X3 represents an amino acid residue selected from Gln and His,
X18 represents Arg,
X20 represents an amino acid residue selected from Lys, Gln, His and (S)MeLys,
X21 represents an amino acid residue selected from Asp and Glu,
X28 represents an amino acid residue selected from Ser and Ala,
X32 represents an amino acid residue selected from Ser and Val.

A further embodiment relates to a group of compounds, wherein
X2 represents an amino acid residue selected from Ser, D-Ser and Aib,
X3 represents Gln,
X18 represents an amino acid residue selected from Arg and Lys,
X20 represents an amino acid residue selected from Lys, Gln, His and (S)MeLys,
X21 represents an amino acid residue selected from Asp and Glu,
X28 represents an amino acid residue selected from Ser and Ala,
X32 represents an amino acid residue selected from Ser and Val.

A further embodiment relates to a group of compounds, wherein
X2 represents an amino acid residue selected from Ser, D-Ser and Aib,
X3 represents His,
X18 represents Arg,
X20 represents Lys,
X21 represents Asp,
X28 represents Ala,
X32 represents an amino acid residue selected from Ser and Val.

A further embodiment relates to a group of compounds, wherein
X2 represents an amino acid residue selected from Ser, D-Ser and Aib,
X3 represents an amino acid residue selected from Gln and His,
X18 represents an amino acid residue selected from Arg and Lys,
X20 represents Lys,
X21 represents an amino acid residue selected from Asp and Glu,
X28 represents an amino acid residue selected from Ser and Ala,
X32 represents an amino acid residue selected from Ser and Val.

A further embodiment relates to a group of compounds, wherein
X2 represents Aib,
X3 represents Gln,
X18 represents Arg,
X20 represents Gln,
X21 represents Asp,
X28 represents an amino acid residue selected from Ser and Ala,
X32 represents Ser.

A further embodiment relates to a group of compounds, wherein
X2 represents an amino acid residue selected from Ser, D-Ser and Aib,
X3 represents Gln,
X18 represents Arg,
X20 represents (S)MeLys,
X21 represents Asp,
X28 represents Ala,
X32 represents an amino acid residue selected from Ser and Val.

A further embodiment relates to a group of compounds, wherein
X2 represents an amino acid residue selected from Ser, D-Ser and Aib,
X3 represents an amino acid residue selected from Gln and His,
X18 represents an amino acid selected from Arg and Lys,
X20 represents an amino acid residue selected from Lys, Gln, His and (S)MeLys,
X21 represents Asp,
X28 represents an amino acid residue selected from Ser and Ala,
X32 represents an amino acid residue selected from Ser and Val.

A further embodiment relates to a group of compounds, wherein
X2 represents an amino acid residue selected from Ser, D-Ser and Aib,
X3 represents Gln,
X18 represents Arg,
X20 represents Lys,
X21 represents Glu,
X28 represents an amino acid residue selected from Ser and Ala,
X32 represents an amino acid residue selected from Ser and Val.

A further embodiment relates to a group of compounds, wherein
X2 represents Aib,
X3 represents Gln,
X18 represents Arg,
X20 represents an amino acid residue selected from Lys and Gln,
X21 represents an amino acid residue selected from Asp and Glu,
X28 represents Ser,
X32 represents Ser.

A further embodiment relates to a group of compounds, wherein
X2 represents an amino acid residue selected from Ser, D-Ser and Aib,
X3 represents an amino acid residue selected from Gln and His,
X18 represents an amino acid selected from Arg and Lys,
X20 represents an amino acid residue selected from Lys, Gln, His and (S)MeLys,
X21 represents an amino acid residue selected from Asp and Glu,
X28 represents an amino acid residue selected from Ser and Ala,
X32 represents Ser.

A further embodiment relates to a group of compounds, wherein
X2 represents an amino acid residue selected from Ser, D-Ser and Aib,
X3 represents an amino acid residue selected from Gln and His,
X18 represents Arg
X20 represents an amino acid residue selected from Lys and (S)MeLys,
X21 represents an amino acid residue selected from Asp and Glu,
X28 represents Ala,
X32 represents Val.

A further embodiment relates to a group of compounds, wherein
X2 represents an amino acid residue selected from Ser and Aib,
X3 represents an amino acid residue selected from Gln and His,
X18 represents Arg,
X20 represents an amino acid residue selected from Lys and Gln,
X21 represents an amino acid residue selected from Asp and Glu,
X28 represents an amino acid residue selected from Ser and Ala,
X32 represents an amino acid residue selected from Ser and Val.

Specific examples of peptidic compounds of formula (I) are the compounds of SEQ ID NO: 5-27, as well as salts and solvates thereof.

Specific examples of peptidic compounds of formula (I) are the compounds of SEQ ID NO: 5-26, as well as salts and solvates thereof.

Specific examples of peptidic compounds of formula (I) are the compounds of SEQ ID NO: 5, 6, 10, 11, 18, as well as salts and solvates thereof.

In certain embodiments, i.e. when the compound of formula (I) comprises genetically encoded amino acid residues, the invention further provides a nucleic acid (which may be DNA or RNA) encoding said compound, an expression vector comprising such a nucleic acid, and a host cell containing such a nucleic acid or expression vector.

In a further aspect, the present invention provides a composition comprising a compound of the invention in admixture with a carrier. In preferred embodiments, the composition is a pharmaceutically acceptable composition and the carrier is a pharmaceutically acceptable carrier. The compound of the invention may be in the form of a salt, e.g. a pharmaceutically acceptable salt or a solvate, e.g. a hydrate. In still a further aspect, the present invention provides a composition for use in a method of medical treatment, particularly in human medicine.

In certain embodiments, the nucleic acid or the expression vector may be used as therapeutic agents, e.g. in gene therapy.

The compounds of formula (I) are suitable for therapeutic application without an additionally therapeutically effective agent. In other embodiments, however, the compounds are used together with at least one additional therapeutically active agent, as described in “combination therapy”.

The compounds of formula (I) are particularly suitable for the treatment or prevention of diseases or disorders caused by, associated with and/or accompanied by disturbances in carbohydrate and/or lipid metabolism, e.g. for the treatment or prevention of hyperglycemia, type 2 diabetes, impaired glucose tolerance, type 1 diabetes, obesity and metabolic syndrome. Further, the compounds of the invention are particularly suitable for the treatment or prevention of degenerative diseases, particularly neurodegenerative diseases.

The compounds described find use, inter alia, in preventing weight gain or promoting weight loss. By “preventing” is meant inhibiting or reducing when compared to the absence of treatment, and is not necessarily meant to imply complete cessation of a disorder.

The compounds of the invention may cause a decrease in food intake and/or increase in energy expenditure, resulting in the observed effect on body weight.

Independently of their effect on body weight, the compounds of the invention may have a beneficial effect on circulating cholesterol levels, being capable of improving lipid levels, particularly LDL, as well as HDL levels (e.g. increasing HDL/LDL ratio).

Thus, the compounds of the invention can be used for direct or indirect therapy of any condition caused or characterised by excess body weight, such as the treatment and/or prevention of obesity, morbid obesity, obesity linked inflammation, obesity linked gallbladder disease, obesity induced sleep apnea. They may also be used for treatment and prevention of the metabolic syndrome, diabetes, hypertension, atherogenic dyslipidemia, atherosclerosis, arteriosclerosis, coronary heart disease, or stroke. Their effects in these conditions may be as a result of or associated with their effect on body weight, or may be independent thereof.

Preferred medical uses include delaying or preventing disease progression in type 2 diabetes, treating metabolic syndrome, treating obesity or preventing overweight, for decreasing food intake, increase energy expenditure, reducing body weight, delaying the progression from impaired glucose tolerance (IGT) to type 2 diabetes; delaying the progression from type 2 diabetes to insulin-requiring diabetes; regulating appetite; inducing satiety; preventing weight regain after successful weight loss; treating a disease or state related to overweight or obesity; treating bulimia; treating binge eating; treating atherosclerosis, hypertension, type 2 diabetes, IGT, dyslipidemia, coronary heart disease, hepatic steatosis, treatment of beta-blocker poisoning, use for inhibition of the motility of the gastrointestinal tract, useful in connection with investigations of the gastrointestinal tract using techniques such as X-ray, CT- and NMR-scanning

Further preferred medical uses include treatment or prevention of degenerative disorders, particularly neurodegenerative disorders such as Alzheimer's disease, Parkinson's disease, Huntington's disease, ataxia, e.g spinocerebellar ataxia, Kennedy disease, myotonic dystrophy, Lewy body dementia, multi-systemic atrophy, amyotrophic lateral sclerosis, primary lateral sclerosis, spinal muscular atrophy, prion-associated diseases, e.g. Creutzfeldt-Jacob disease, multiple sclerosis, telangiectasia, Batten disease, corticobasal degeneration, corticobasal degeneration, subacute combined degeneration of spinal cord, Tabes dorsalis, Tay-Sachs disease, toxic encephalopathy, infantile Refsum disease, Refsum disease, neuroacanthocytosis, Niemann-Pick disease, Lyme disease, Machado-Joseph disease, Sandhoff disease, Shy-Drager syndrome, wobbly hedgehog syndrome, proteopathy, cerebral β-amyloid angiopathy, retinal ganglion cell degeneration in glaucoma, synucleinopathies, tauopathies, frontotemporal lobar degeneration (FTLD), dementia, cadasil syndrome, hereditary cerebral hemorrhage with amyloidosis, Alexander disease, seipinopathies, familial amyloidotic neuropathy, senile systemic amyloidosis, serpinopathies, AL (light chain) amyloidosis (primary systemic amyloidosis), AH (heavy chain) amyloidosis, AA (secondary) amyloidosis, aortic medial amyloidosis, ApoAI amyloidosis, ApoAII amyloidosis, ApoAIV amyloidosis, familial amyloidosis of the Finnish type (FAF), Lysozyme amyloidosis, Fibrinogen amyloidosis, Dialysis amyloidosis, Inclusion body myositis/myopathy, Cataracts, Retinitis pigmentosa with rhodopsin mutations, medullary thyroid carcinoma, cardiac atrial amyloidosis, pituitary prolactinoma, Hereditary lattice corneal dystrophy, Cutaneous lichen amyloidosis, Mallory bodies, corneal lactoferrin amyloidosis, pulmonary alveolar proteinosis, odontogenic (Pindborg) tumor amyloid, cystic fibrosis, sickle cell disease or critical illness myopathy (CIM).

Further medical uses include treatment of hyperglycemia, type 2 diabetes, obesity, particularly Type 2 diabetes.

### DETAILED DESCRIPTION OF THE INVENTION

### Definitions

The amino acid sequences of the present invention contain the conventional one letter and three letter codes for naturally occurring amino acids, as well as generally accepted three letter codes for other amino acids, such as Aib (α-aminoisobutyric acid).

Furthermore, the following code was used for the amino acid shown in Table 1a:

TABLE 1

| Name | Structure | Code |
|---|---|---|
| (S)- -methyl- lysine | 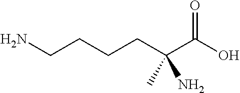 | (S)MeLys |

The term “native exendin-4” refers to native exendin-4 having the sequence

(SEQ ID NO: 1)
HGEGTFTSDLSKQMEEEAVRLFIEWLKNGGPSSGAPPPS-NH₂.

The invention provides peptidic compounds as defined above.

The peptidic compounds of the present invention comprise a linear backbone of amino carboxylic acids linked by peptide, i.e. carboxamide bonds. Preferably, the amino carboxylic acids are α-amino carboxylic acids and more preferably L-α-amino carboxylic acids, unless indicated otherwise. The peptidic compounds preferably comprise a backbone sequence of 39 amino carboxylic acids.

For the avoidance of doubt, in the definitions provided herein, it is generally intended that the sequence of the peptidic moiety (II) differs from native exendin-4 at least at one of those positions which are stated to allow variation. Amino acids within the peptide moiety (II) can be considered to be numbered consecutively from 1 to 39 in the conventional N-terminal to C-terminal direction. Reference to a “position” within peptidic moiety (II) should be constructed accordingly, as should reference to positions within native exendin-4 and other molecules.

In a further aspect, the present invention provides a composition comprising a compound of the invention as described herein, or a salt or solvate thereof, in admixture with a carrier.

The invention also provides the use of a compound of the present invention for use as a medicament, particularly for the treatment of a condition as described below.

The invention also provides a composition wherein the composition is a pharmaceutically acceptable composition, and the carrier is a pharmaceutically acceptable carrier.

Peptide Synthesis

The skilled person is aware of a variety of different methods to prepare peptides that are described in this invention. These methods include but are not limited to synthetic approaches and recombinant gene expression. Thus, one way of preparing these peptides is the synthesis in solution or on a solid support and subsequent isolation and purification. A different way of preparing the peptides is gene expression in a host cell in which a DNA sequence encoding the peptide has been introduced. Alternatively, the gene expression can be achieved without utilizing a cell system. The methods described above may also be combined in any way.

A preferred way to prepare the peptides of the present invention is solid phase synthesis on a suitable resin. Solid phase peptide synthesis is a well-established methodology (see for example: Stewart and Young, Solid Phase Peptide Synthesis, Pierce Chemical Co., Rockford, Ill., 1984; E. Atherton and R. C. Sheppard, Solid Phase Peptide Synthesis. A Practical Approach, Oxford-IRL Press, New York, 1989). Solid phase synthesis is initiated by attaching an N-terminally protected amino acid with its carboxy terminus to an inert solid support carrying a cleavable linker. This solid support can be any polymer that allows coupling of the initial amino acid, e.g. a trityl resin, a chlorotrityl resin, a Wang resin or a Rink resin in which the linkage of the carboxy group (or carboxamide for Rink resin) to the resin is sensitive to acid (when Fmoc strategy is used). The polymer support must be stable under the conditions used to deprotect the α-amino group during the peptide synthesis.

After the first amino acid has been coupled to the solid support, the α-amino protecting group of this amino acid is removed. The remaining protected amino acids are then coupled one after the other in the order represented by the peptide sequence using appropriate amide coupling reagents, for example BOP (benzotriazol-1-yl-oxy-tris-(dimethylamino)-phosphonium), HBTU (2-(1H-benzotriazol-1-yl)-1,1,3,3-tetramethyl-uronium), HATU (O-(7-azabenztriazol-1-yl-oxy-tris-(dimethylamino)-phosphonium) or DIC (N,N′-diisopropylcarbodiimide)/HOBt (1-hydroxybenzotriazol), wherein BOP, HBTU and HATU are used with tertiary amine bases. Alternatively, the liberated N-terminus can be functionalized with groups other than amino acids, for example carboxylic acids, etc.

Usually, reactive side-chain groups of the amino acids are protected with suitable blocking groups. These protecting groups are removed after the desired peptides have been assembled. They are removed concomitantly with the cleavage of the desired product from the resin under the same conditions. Protecting groups and the procedures to introduce protecting groups can be found in Protective Groups in Organic Synthesis, 3d ed., Greene, T. W. and Wuts, P. G. M., Wiley & Sons (New York: 1999).

In some cases it might be desirable to have side-chain protecting groups that can selectively be removed while other side-chain protecting groups remain intact. In this case the liberated functionality can be selectively functionalized. For example, a lysine may be protected with an ivDde protecting group (S. R. Chhabra et al., Tetrahedron Lett. 39, (1998), 1603) which is labile to a very nucleophilic base, for example 4% hydrazine in DMF (dimethyl formamide). Thus, if the N-terminal amino group and all side-chain functionalities are protected with acid labile protecting groups, the ivDde ([1-(4,4-dimethyl-2,6-dioxocyclohex-1-ylidene)-3-methylbutyl) group can be selectively removed using 4% hydrazine in DMF and the corresponding free amino group can then be further modified, e.g. by acylation. The lysine can alternatively be coupled to a protected amino acid and the amino group of this amino acid can then be deprotected resulting in another free amino group which can be acylated or attached to further amino acids.

Finally the peptide is cleaved from the resin. This can be achieved by using King's cocktail (D. S. King, C. G. Fields, G. B. Fields, Int. J. Peptide Protein Res. 36, 1990, 255-266). The raw material can then be purified by chromatography, e.g. preparative RP-HPLC, if necessary.

Potency

As used herein, the term “potency” or “in vitro potency” is a measure for the ability of a compound to activate the receptors for GLP-1, glucagon or optionally GIP in a cell-based assay. Numerically, it is expressed as the “EC50 value”, which is the effective concentration of a compound that induces a half maximal increase of response (e.g. formation of intracellular cAMP) in a dose-response experiment.

Therapeutic Uses

According to one aspect, the compounds of the invention are for use in medicine, particularly human medicine.

The compounds of the invention are agonists for the receptors for GLP-1 and for glucagon as well as optionally for GIP (e.g. “dual or trigonal agonists”) and may provide an attractive option for targeting the metabolic syndrome by allowing simultaneous treatment of obesity and diabetes.

Metabolic syndrome is a combination of medical disorders that, when occurring together, increase the risk of developing type 2 diabetes, as well as atherosclerotic vascular disease, e.g. heart disease and stroke. Defining medical parameters for the metabolic syndrome include diabetes mellitus, impaired glucose tolerance, raised fasting glucose, insulin resistance, urinary albumin secretion, central obesity, hypertension, elevated triglycerides, elevated LDL cholesterol and reduced HDL cholesterol.

Obesity is a medical condition in which excess body fat has accumulated to the extent that it may have an adverse effect on health and life expectancy and due to its increasing prevalence in adults and children it has become one of the leading preventable causes of death in modem world. It increases the likelihood of various other diseases, including heart disease, type 2 diabetes, obstructive sleep apnea, certain types of cancer, as well as osteoarthritis, and it is most commonly caused by a combination of excess food intake, reduced energy expenditure, as well as genetic susceptibility.

Diabetes mellitus, often simply called diabetes, is a group of metabolic diseases in which a person has high blood sugar levels, either because the body does not produce enough insulin, or because cells do not respond to the insulin that is produced. The most common types of diabetes are: (1) type 1 diabetes, where the body fails to produce insulin; (2) type 2 diabetes, where the body fails to use insulin properly, combined with an increase in insulin deficiency over time, and (3) gestational diabetes, where women develop diabetes due to their pregnancy. All forms of diabetes increase the risk of long-term complications, which typically develop after many years. Most of these long-term complications are based on damage to blood vessels and can be divided into the two categories “macrovascular” disease, arising from atherosclerosis of larger blood vessels and “microvascular” disease, arising from damage of small blood vessels. Examples for macrovascular disease conditions are ischemic heart disease, myocardial infarction, stroke and peripheral vascular disease. Examples for microvascular diseases are diabetic retinopathy, diabetic nephropathy, as well as diabetic neuropathy.

The receptors for GLP-1, glucagon and GIP are members of the family B of G-protein coupled receptors. They are highly related to each other and share not only a significant level of sequence identity, but have also similar mechanisms of ligand recognition and intracellular signaling pathways.

Similarly, the peptides GLP-1, GIP and glucagon share regions of high sequence identity/similarity. GLP-1 and glucagon are produced from a common precursor, preproglucagon, which is differentially processed in a tissue-specific manner to yield e.g. GLP-1 in intestinal endocrine cells and glucagon in alpha cells of pancreatic islets. GIP is derived from a larger proGIP prohormone precursor and is synthesized and released from K-cells located in the small intestine.

The peptidic incretin hormones GLP-1 and GIP are secreted by intestinal endocrine cells in response to food and account for up to 70% of meal-stimulated insulin secretion. Evidence suggests that GLP-1 secretion is reduced in subjects with impaired glucose tolerance or type 2 diabetes, whereas responsiveness to GLP-1 is still preserved in these patients. Thus, targeting of the GLP-1 receptor with suitable agonists offers an attractive approach for treatment of metabolic disorders, including diabetes. The receptor for GLP-1 is distributed widely, being found mainly in pancreatic islets, brain, heart, kidney and the gastrointestinal tract. In the pancreas, GLP-1 acts in a strictly glucose-dependent manner by increasing secretion of insulin from beta cells. This glucose-dependency shows that activation of GLP-1 receptors is unlikely to cause hypoglycemia. Also the receptor for GIP is broadly expressed in peripheral tissues including pancreatic islets, adipose tissue, stomach, small intestine, heart, bone, lung, kidney, testis, adrenal cortex, pituitary, endothelial cells, trachea, spleen, thymus, thyroid and brain.

Consistent with its biological function as incretin hormone, the pancreatic beta cell express the highest levels of the receptor for GIP in humans. There is some clinical evidence that the GIP-receptor mediated signaling could be impaired in patients with T2DM but the impairment of GIP-action is shown to be reversible and could be restored with improvement of the diabetic status. Of note, the stimulation of insulin secretion by both incretin hormones, GIP and GLP-1, is strictly glucose-dependent ensuring a fail-safe mechanism associated with a low risk for hypoglycemia.

At the beta cell level, GLP-1 and GIP have been shown to promote glucose sensitivity, neogenesis, proliferation, transcription of proinsulin and hypertrophy, as well as antiapoptosis. A peptide with dual agonistic activity for the GLP-1 and the GIP receptor could be anticipated to have additive or synergistic anti-diabetic benefit. Other relevant effects of GLP-1 beyond the pancreas include delayed gastric emptying, increased satiety, decreased food intake, reduction of body weight, as well as neuroprotective and cardioprotective effects. In patients with type 2 diabetes, such extrapancreatic effects could be particularly important considering the high rates of comorbidities like obesity and cardiovascular disease. Further GIP actions in peripheral tissues beyond the pancreas comprise increased bone formation and decreased bone resorption as well as neuroprotective effects which might be beneficial for the treatment of osteoporosis and cognitive defects like Alzheimer's disease.

Glucagon is a 29-amino acid peptide hormone that is produced by pancreatic alpha cells and released into the bloodstream when circulating glucose is low. An important physiological role of glucagon is to stimulate glucose output in the liver, which is a process providing the major counterregulatory mechanism for insulin in maintaining glucose homeostasis in vivo.

Glucagon receptors are however also expressed in extrahepatic tissues such as kidney, heart, adipocytes, lymphoblasts, brain, retina, adrenal gland and gastrointestinal tract, suggesting a broader physiological role beyond glucose homeostasis. Accordingly, recent studies have reported that glucagon has therapeutically positive effects on energy management, including stimulation of energy expenditure and thermogenesis, accompanied by reduction of food intake and body weight loss. Altogether, stimulation of glucagon receptors might be useful in the treatment of obesity and the metabolic syndrome.

Oxyntomodulin is a peptide hormone consisting of glucagon with a C-terminal extension encompassing eight amino acids. Like GLP-1 and glucagon, it is preformed in preproglucagon and cleaved and secreted in a tissue-specific manner by endocrinal cells of the small bowel. Oxyntomodulin is known to stimulate both the receptors for GLP-1 and glucagon and is therefore the prototype of a dual agonist.

As GLP-1 and GIP are known for its anti-diabetic effects, GLP-1 and glucagon are both known for their food intake-suppressing effects and glucagon is also a mediator of additional energy expenditure, it is conceivable that a combination of the activities of the two hormones in one molecule can yield a powerful medication for treatment of the metabolic syndrome and in particular its components diabetes and obesity.

Accordingly, the compounds of the invention may be used for treatment of glucose intolerance, insulin resistance, pre-diabetes, increased fasting glucose, type 2 diabetes, hypertension, dyslipidemia, arteriosclerosis, coronary heart disease, peripheral artery disease, stroke or any combination of these individual disease components.

In addition, they may be used for control of appetite, feeding and calorie intake, increase of energy expenditure, prevention of weight gain, promotion of weight loss, reduction of excess body weight and altogether treatment of obesity, including morbid obesity.

Further disease states and health conditions which could be treated with the compounds of the invention are obesity-linked inflammation, obesity-linked gallbladder disease and obesity-induced sleep apnea.

Although all these conditions could be associated directly or indirectly with obesity, the effects of the compounds of the invention may be mediated in whole or in part via an effect on body weight, or independent thereof.

Further diseases to be treated are neurodegenerative diseases such as Alzheimer's disease or Parkinson's disease, or other degenerative diseases as described above.

Compared to GLP-1, glucagon and oxyntomodulin, exendin-4 has beneficial physicochemical properties, such as solubility and stability in solution and under physiological conditions (including enzymatic stability towards degradation by enzymes, such as DPP-4 or NEP), which results in a longer duration of action in vivo. Therefore, exendin-4 might serve as good starting scaffold to obtain exendin-4 analogues with dual or even triple pharmacologies, e.g., GLP-1/Glucagon and optionally in addition GIP agonism.

Nevertheless, also exendin-4 has been shown to be chemically labile due to methionine oxidation in position 14 as well as deamidation and isomerization of asparagine in position 28. Therefore, stability might be further improved by substitution of methionine at position 14 and the avoidance of sequences that are known to be prone to degradation via aspartimide formation, especially Asp-Gly or Asn-Gly at positions 28 and 29.

Pharmaceutical Compositions

The term “pharmaceutical composition” indicates a mixture containing ingredients that are compatible when mixed and which may be administered. A pharmaceutical composition may include one or more medicinal drugs. Additionally, the pharmaceutical composition may include carriers, buffers, acidifying agents, alkalizing agents, solvents, adjuvants, tonicity adjusters, emollients, expanders, preservatives, physical and chemical stabilizers e.g. surfactants, antioxidants and other components, whether these are considered active or inactive ingredients. Guidance for the skilled in preparing pharmaceutical compositions may be found, for example, in Remington: The Science and Practice of Pharmacy, (20th ed.) ed. A. R. Gennaro A. R., 2000, Lippencott Williams & Wilkins and in R. C. Rowe et al (Ed), Handbook of Pharmaceutical Excipients, PhP, May 2013 update.

The exendin-4 peptide derivatives of the present invention, or salts thereof, are administered in conjunction with an acceptable pharmaceutical carrier, diluent, or excipient as part of a pharmaceutical composition. A “pharmaceutically acceptable carrier” is a carrier which is physiologically acceptable (e.g. physiologically acceptable pH) while retaining the therapeutic properties of the substance with which it is administered. Standard acceptable pharmaceutical carriers and their formulations are known to one skilled in the art and described, for example, in Remington: The Science and Practice of Pharmacy, (20th ed.) ed. A. R. Gennaro A. R., 2000, Lippencott Williams & Wilkins and in R. C. Rowe et al (Ed), Handbook of Pharmaceutical excipients, PhP, May 2013 update. One exemplary pharmaceutically acceptable carrier is physiological saline solution.

In one embodiment carriers are selected from the group of buffers (e.g. citrate/citric acid), acidifying agents (e.g. hydrochloric acid), alkalizing agents (e.g. sodium hydroxide), preservatives (e.g. phenol), co-solvents (e.g. polyethylene glycol 400), tonicity adjusters (e.g. mannitol), stabilizers (e.g. surfactant, antioxidants, amino acids).

Concentrations used are in a range that is physiologically acceptable.

Acceptable pharmaceutical carriers or diluents include those used in formulations suitable for oral, rectal, nasal or parenteral (including subcutaneous, intramuscular, intravenous, intradermal, and transdermal) administration. The compounds of the present invention will typically be administered parenterally.

The term “pharmaceutically acceptable salt” means salts of the compounds of the invention which are safe and effective for use in mammals. Pharmaceutically acceptable salts may include, but are not limited to, acid addition salts and basic salts. Examples of acid addition salts include chloride, sulfate, hydrogen sulfate, (hydrogen) phosphate, acetate, citrate, tosylate or mesylate salts. Examples of basic salts include salts with inorganic cations, e.g. alkaline or alkaline earth metal salts such as sodium, potassium, magnesium or calcium salts and salts with organic cations such as amine salts. Further examples of pharmaceutically acceptable salts are described in Remington: The Science and Practice of Pharmacy, (20th ed.) ed. A. R. Gennaro A. R., 2000, Lippencott Williams & Wilkins or in Handbook of Pharmaceutical Salts, Properties, Selection and Use, e.d. P. H. Stahl, C. G. Wermuth, 2002, jointly published by Verlag Helvetica Chimica Acta, Zurich, Switzerland, and Wiley-VCH, Weinheim, Germany.

The term “solvate” means complexes of the compounds of the invention or salts thereof with solvent molecules, e.g. organic solvent molecules and/or water.

In the pharmaceutical composition, the exendin-4 derivative can be in monomeric or oligomeric form.

The term “therapeutically effective amount” of a compound refers to a nontoxic but sufficient amount of the compound to provide the desired effect. The amount of a compound of the formula I necessary to achieve the desired biological effect depends on a number of factors, for example the specific compound chosen, the intended use, the mode of administration and the clinical condition of the patient. An appropriate “effective” amount in any individual case may be determined by one of ordinary skill in the art using routine experimentation For example the “therapeutically effective amount” of a compound of the formula (I) is about 0.01 to 50 mg/dose, preferably 0.1 to 10 mg/dose.

Pharmaceutical compositions of the invention are those suitable for parenteral (for example subcutaneous, intramuscular, intradermal or intravenous), oral, rectal, topical and peroral (for example sublingual) administration, although the most suitable mode of administration depends in each individual case on the nature and severity of the condition to be treated and on the nature of the compound of formula I used in each case.

Suitable pharmaceutical compositions may be in the form of separate units, for example capsules, tablets and powders in vials or ampoules, each of which contains a defined amount of the compound; as powders or granules; as solution or suspension in an aqueous or nonaqueous liquid; or as an oil-in-water or water-in-oil emulsion. It may be provided in single dose injectable form, for example in the form of a pen. The compositions may, as already mentioned, be prepared by any suitable pharmaceutical method which includes a step in which the active ingredient and the carrier (which may consist of one or more additional ingredients) are brought into contact.

In certain embodiments the pharmaceutical composition may be provided together with a device for application, for example together with a syringe, an injection pen or an autoinjector. Such devices may be provided separate from a pharmaceutical composition or prefilled with the pharmaceutical composition.

Combination Therapy

The compounds of the present invention, dual agonists for the GLP-1 and glucagon receptors, can be widely combined with other pharmacologically active compounds, such as all drugs mentioned in the Rote Liste 2013, e.g. with all weight-reducing agents or appetite suppressants mentioned in the Rote Liste 2013, chapter 1, all lipid-lowering agents mentioned in the Rote Liste 2013, chapter 58, all antihypertensives and nephroprotectives, mentioned in the Rote Liste 2013, or all diuretics mentioned in the Rote Liste 2013, chapter 36.

The active ingredient combinations can be used especially for a synergistic improvement in action. They can be applied either by separate administration of the active ingredients to the patient or in the form of combination products in which a plurality of active ingredients are present in one pharmaceutical preparation. When the active ingredients are administered by separate administration of the active ingredients, this can be done simultaneously or successively.

Most of the active ingredients mentioned hereinafter are disclosed in the USP Dictionary of USAN and International Drug Names, US Pharmacopeia, Rockville 2011.

Other active substances which are suitable for such combinations include in particular those which for example potentiate the therapeutic effect of one or more active substances with respect to one of the indications mentioned and/or which allow the dosage of one or more active substances to be reduced.

Therapeutic agents which are suitable for combinations include, for example, antidiabetic agents such as:

Insulin and Insulin derivatives, for example: Glargine/Lantus® , 270-330 U/mL of insulin glargine (EP 2387989 A), 300 U/mL of insulin glargine (EP 2387989 A), Glulisin/Apidra®, Detemir/Levemir®, Lispro/Humalog®/Liprolog®, Degludec/DegludecPlus, Aspart, basal insulin and analogues (e.g. LY-2605541, LY2963016, NN1436), PEGylated insulin Lispro, Humulin®, Linjeta, SuliXen®, NN1045, Insulin plus Symlin, PE0139, fast-acting and short-acting insulins (e.g. Linjeta, PH20, NN1218, HinsBet), (APC-002)hydrogel, oral, inhalable, transdermal and sublingual insulins (e.g. Exubera®, Nasulin®, Afrezza, Tregopil, TPM 02, Capsulin, Oral-Lyn®, Cobalamin® oral insulin, ORMD-0801, NN1953, NN1954, NN1956, VIAtab, Oshadi oral insulin). Additionally included are also those insulin derivatives which are bonded to albumin or another protein by a bifunctional linker.

GLP-1, GLP-1 analogues and GLP-1 receptor agonists, for example: Lixisenatide/AVE0010/ZP10/Lyxumia, Exenatide/Exendin-4/Byetta/Bydureon/ITCA 650/AC-2993, Liraglutide/Victoza, Semaglutide, Taspoglutide, Syncria/Albiglutide, Dulaglutide, rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

DPP-4 inhibitors, for example: Alogliptin/Nesina, Trajenta/Linagliptin/BI-1356/Ondero/Trajenta/Tradjenta/Trayenta/Tradzenta, Saxagliptin/Onglyza, Sitagliptin/Januvia/Xelevia/Tesave/Janumet/Velmetia, Galvus/Vildagliptin, Anagliptin, Gemigliptin, Teneligliptin, Melogliptin, Trelagliptin, DA-1229, Omarigliptin/MK-3102, KM-223, Evogliptin, ARI-2243, PBL-1427, Pinoxacin.

SGLT2 inhibitors, for example: Invokana/Canaglifozin, Forxiga/Dapagliflozin, Remoglifozin, Sergliflozin, Empagliflozin, Ipragliflozin, Tofogliflozin, Luseogliflozin, LX-4211, Ertuglifozin/PF-04971729, RO-4998452, EGT-0001442, KGA-3235/DSP-3235, LIK066, SBM-TFC-039,

Biguanides (e.g. Metformin, Buformin, Phenformin), Thiazolidinediones (e.g. Pioglitazone, Rivoglitazone, Rosiglitazone, Troglitazone), dual PPAR agonists (e.g. Aleglitazar, Muraglitazar, Tesaglitazar), Sulfonylureas (e.g. Tolbutamide, Glibenclamide, Glimepiride/Amaryl, Glipizide), Meglitinides (e.g. Nateglinide, Repaglinide, Mitiglinide), Alpha-glucosidase inhibitors (e.g. Acarbose, Miglitol, Voglibose), Amylin and Amylin analogues (e.g. Pramlintide, Symlin).

GPR119 agonists (e.g. GSK-263A, PSN-821, MBX-2982, APD-597, ZYG-19, DS-8500), GPR40 agonists (e.g. Fasiglifam/TAK-875, TUG-424, P-1736, JTT-851, GW9508).

Other suitable combination partners are: Cycloset, inhibitors of 11-beta-HSD (e.g. LY2523199, BMS770767, RG-4929, BMS816336, AZD-8329, HSD-016, BI-135585), activators of glucokinase (e.g. TTP-399, AMG-151, TAK-329, GKM-001), inhibitors of DGAT (e.g. LCQ-908), inhibitors of protein tyrosinephosphatase 1 (e.g. Trodusquemine), inhibitors of glucose-6-phosphatase, inhibitors of fructose-1,6-bisphosphatase, inhibitors of glycogen phosphorylase, inhibitors of phosphoenol pyruvate carboxykinase, inhibitors of glycogen synthase kinase, inhibitors of pyruvate dehydrokinase, alpha2-antagonists, CCR-2 antagonists, SGLT-1 inhibitors (e.g. LX-2761).

One or more lipid lowering agents are also suitable as combination partners, such as for example: HMG-CoA-reductase inhibitors (e.g. Simvastatin, Atorvastatin), fibrates (e.g. Bezafibrate, Fenofibrate), nicotinic acid and the derivatives thereof (e.g. Niacin), PPAR-(alpha, gamma or alpha/gamma) agonists or modulators (e.g. Aleglitazar), PPAR-delta agonists, ACAT inhibitors (e.g. Avasimibe), cholesterol absorption inhibitors (e.g. Ezetimibe), Bile acid-binding substances (e.g. Cholestyramine), ileal bile acid transport inhibitors, MTP inhibitors, or modulators of PCSK9.

HDL-raising compounds such as: CETP inhibitors (e.g. Torcetrapib, Anacetrapid, Dalcetrapid, Evacetrapid, JTT-302, DRL-17822, TA-8995) or ABC1 regulators.

Other suitable combination partners are one or more active substances for the treatment of obesity, such as for example: Sibutramine, Tesofensine, Orlistat, antagonists of the cannabinoid-1 receptor, MCH-1 receptor antagonists, MC4 receptor agonists, NPY5 or NPY2 antagonists (e.g. Velneperit), beta-3-agonists, leptin or leptin mimetics, agonists of the 5HT2c receptor (e.g. Lorcaserin), or the combinations of bupropione/naltrexone, bupropione/zonisamide, bupropione/phentermine or pramlintide/metreleptin.

Other suitable combination partners are:

Further gastrointestinal peptides such as Peptide YY 3-36 (PYY3-36) or analogues thereof, pancreatic polypeptide (PP) or analogues thereof.

Glucagon receptor agonists or antagonists, GIP receptor agonists or antagonists, ghrelin antagonists or inverse agonists, Xenin and analogues thereof.

Moreover, combinations with drugs for influencing high blood pressure, chronic heart failure or atherosclerosis, such as e.g.: Angiotensin II receptor antagonists (e.g. telmisartan, candesartan, valsartan, losartan, eprosartan, irbesartan, olmesartan, tasosartan, azilsartan), ACE inhibitors, ECE inhibitors, diuretics, beta-blockers, calcium antagonists, centrally acting hypertensives, antagonists of the alpha-2-adrenergic receptor, inhibitors of neutral endopeptidase, thrombocyte aggregation inhibitors and others or combinations thereof are suitable.

In another aspect, this invention relates to the use of a compound according to the invention or a physiologically acceptable salt thereof combined with at least one of the active substances described above as a combination partner, for preparing a medicament which is suitable for the treatment or prevention of diseases or conditions which can be affected by binding to the receptors for GLP-1 and glucagon and by modulating their activity. This is preferably a disease in the context of the metabolic syndrome, particularly one of the diseases or conditions listed above, most particularly diabetes or obesity or complications thereof.

The use of the compounds according to the invention, or a physiologically acceptable salt thereof, in combination with one or more active substances may take place simultaneously, separately or sequentially.

The use of the compound according to the invention, or a physiologically acceptable salt thereof, in combination with another active substance may take place simultaneously or at staggered times, but particularly within a short space of time. If they are administered simultaneously, the two active substances are given to the patient together; if they are used at staggered times, the two active substances are given to the patient within a period of less than or equal to 12 hours, but particularly less than or equal to 6 hours.

Consequently, in another aspect, this invention relates to a medicament which comprises a compound according to the invention or a physiologically acceptable salt of such a compound and at least one of the active substances described above as combination partners, optionally together with one or more inert carriers and/or diluents.

The compound according to the invention, or physiologically acceptable salt or solvate thereof, and the additional active substance to be combined therewith may both be present together in one formulation, for example a tablet or capsule, or separately in two identical or different formulations, for example as so-called kit-of-parts.

### LEGENDS TO THE FIGURES

FIG. 1. Effect of treatment with SEQ ID NO: 10 at 100 μg/kg, s.c. on glucose lowering in non-fasted female diabetic dbdb-mice, represented as change from baseline. Data are mean+S

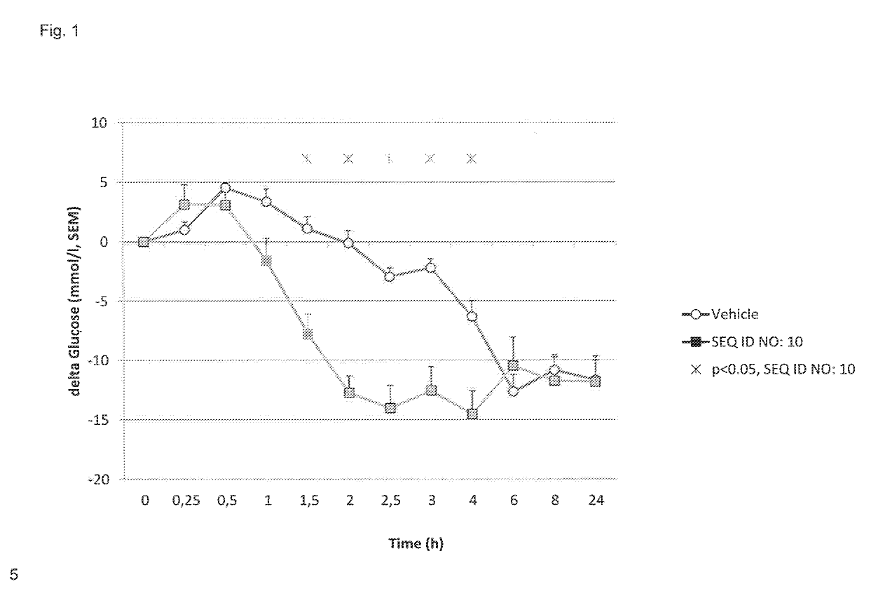

### METHODS

Abbreviations employed are as follows:
AA amino acid
cAMP cyclic adenosine monophosphate
Boc tert-butyloxycarbonyl
BOP (benzotriazol-1-yloxy)tris(dimethylamino)phosphonium hexafluorophosphate
BSA bovine serum albumin
tBu tertiary butyl
Dde 1-(4,4-dimethyl-2,6-dioxocyclohexylidene)-ethyl
ivDde 1-(4,4-dimethyl-2,6-dioxocyclohexylidene)3-methyl-butyl
DIC N,N′-diisopropylcarbodiimide
DIPEA N,N-diisopropylethylamine
DMEM Dulbecco's modified Eagle's medium
DMF dimethyl formamide
EDT ethanedithiol
FBS fetal bovine serum
Fmoc fluorenylmethyloxycarbonyl
HATU O-(7-azabenzotriazol-1-yl)-N,N,N′,N′-tetramethyluronium hexafluorophosphate
HBSS Hanks' Balanced Salt Solution
HBTU 2-(1H-benzotriazol-1-yl)-1,1,3,3-tetramethyl-uronium hexafluorophosphate
HEPES 2-[4-(2-hydroxyethyl)piperazin-1-yl]ethanesulfonic acid
HOBt 1-hydroxybenzotriazole
HOSu N-hydroxysuccinimide
HPLC High Performance Liquid Chromatography
HTRF Homogenous Time Resolved Fluorescence
IBMX 3-isobutyl-1-methylxanthine
LC/MS Liquid Chromatography/Mass Spectrometry
Palm palmitoyl
PBS phosphate buffered saline
PEG polyethylene glycole
PK pharmacokinetic
RP-HPLC reversed-phase high performance liquid chromatography
TFA trifluoroacetic acid
Trt trityl
UPLC Ultra Performance Liquid Chromatography
UV ultraviolet

General Synthesis of Peptidic Compounds

Materials:

Different Rink-Amide resins (4-(2′,4′-Dimethoxyphenyl-Fmoc-aminomethyl)-phenoxyacetamido-norleucylaminomethyl resin, Merck Biosciences; 4-[(2,4-Dimethoxyphenyl)(Fmoc-amino)methyl]phenoxy acetamido methyl resin, Agilent Technologies) were used for the synthesis of peptide amides with loadings in the range of 0.3-0.4 mmol/g.

Fmoc protected natural amino acids were purchased from Protein Technologies Inc., Senn Chemicals, Merck Biosciences, Novabiochem, his Biotech, Nagase or Bachem. The following standard amino acids were used throughout the syntheses: Fmoc-L-Ala-OH, Fmoc-L-Arg(Pbf)-OH, Fmoc-L-Asn(Trt)-OH, Fmoc-L-Asp(OtBu)-OH, Fmoc-L-Cys(Trt)-OH, Fmoc-L-Gln(Trt)-OH, Fmoc-L-Glu(OtBu)-OH, Fmoc-Gly-OH, Fmoc-L-His(Trt)-OH, Fmoc-L-Ile-OH, Fmoc-L-Leu-OH, Fmoc-L-Lys(Boc)-OH, Fmoc-L-Met-OH, Fmoc-L-Phe-OH, Fmoc-L-Pro-OH, Fmoc-L-Ser(tBu)-OH, Fmoc-L-Thr(tBu)-OH, Fmoc-L-Trp(Boc)-OH, Fmoc-L-Tyr(tBu)-OH, Fmoc-L-Val-OH.

In addition, the following special amino acids were purchased from the same suppliers as above: Fmoc-L-Lys(ivDde)-OH, Fmoc-Aib-OH, Fmoc-D-Ser(tBu)-OH, Fmoc-D-Ala-OH, Boc-L-His(Boc)-OH (available as toluene solvate) and Boc-L-His(Trt)-OH, Fmoc-L-Nle-OH, Fmoc-L-Met(O)—OH, Fmoc-L-Met(O2)-OH, Fmoc-(S)MeLys(Boc)-OH, Fmoc-(R)MeLys(Boc)-OH, Fmoc-(S)MeOrn(Boc)-OH and Boc-L-Tyr(tBu)-OH.

The solid phase peptide syntheses were performed for example on a Prelude Peptide Synthesizer (Protein Technologies Inc) or similar automated synthesizer using standard Fmoc chemistry and HBTU/DIPEA activation. DMF was used as the solvent. Deprotection: 20% piperidine/DMF for 2×2.5 min. Washes: 7×DME Coupling 2:5:10 200 mM AA/500 mM HBTU/2M DIPEA in DMF 2× for 20 min. Washes: 5×DMF.

All the peptides that had been synthesized were cleaved from the resin with King's cleavage cocktail consisting of 82.5% TFA, 5% phenol, 5% water, 5% thioanisole, 2.5% EDT. The crude peptides were then precipitated in diethyl or diisopropyl ether, centrifuged, and lyophilized Peptides were analyzed by analytical HPLC and checked by ESI mass spectrometry. Crude peptides were purified by a conventional preparative HPLC purification procedure.

Analytical HPLC/UPLC

Method A: detection at 215 nm
column: Aeris Peptide, 3.6 μm, XB-C18 (250×4 6 mm) at 60° C.
solvent: H₂O+0.1% TFA: ACN+0.1% TFA (flow 1.5 ml/min)
gradient: 90:10 (0 min) to 90:10 (3 min) to 10:90 (43 min) to 10:90 (48 min) to 90:10 (49 min) to 90:10 (50 min)

Method B: detection at 220 nm
column: Zorbax, 5 μm, C18 (250×4 6 mm) at 25° C.
solvent: H₂O+0.1% TFA: 90% ACN+10% H₂O+0.1% TFA (flow 1.0 ml/min)
gradient: 100:0 (0 min) to 98:2 (2 min) to 30:70 (15 min) to 5:95 (20 min) to 0:100 (25 min) to 0:100 (30 min) to 98:2 (32 min) to 98:2 (35 min)

Method C1: detection at 210-225 nm, optionally coupled to a mass analyser Waters LCT Premier, electrospray positive ion mode
column: Waters ACQUITY UPLC® BEH™ C18 1.7 μm (150×2.1 mm) at 50° C.
solvent: H₂O+1% FA: ACN+1% FA (flow 0.5 ml/min)
gradient: 95:5 (0 min) to 95:5 (1.80 min) to 80:20 (1.85 min) to 80:20 (3 min) to 60:40 (23 min) to 25:75 (23.1 min) to 25:75 (25 min) to 95:5 (25.1 min) to 95:5 (30 min)

Method C2: detection at 210-225 nm, optionally coupled to a mass analyser Waters LCT Premier, electrospray positive ion mode
column: Waters ACQUITY UPLC® BEH™ C18 1.7 μm (150×2.1 mm) at 50° C.
solvent: H₂O+1% FA: ACN+1% FA (flow 0.6 ml/min)
gradient: 95:5 (0 min) to 95:5 (1 min) to 65:35 (2 min) to 65:35 (3 min) to 45:55 (23 min) to 25:75 (23.1 min) to 25:75 (25 min) to 95:5 (25.1 min) to 95:5 (30 min)

Method C3: detection at 210-225 nm, optionally coupled to a mass analyser Waters LCT Premier, electrospray positive ion mode
column: Waters ACQUITY UPLC® BEH™ C18 1.7 μm (150×2.1 mm) at 50° C.
solvent: H₂O+1% FA: ACN+1% FA (flow 1 ml/min)
gradient: 95:5 (0 min) to 95:5 (1 min) to 65:35 (2 min) to 65:35 (3 min) to 45:55 (20 min) to 2:98 (20.1 min) to 2:98 (25 min) to 95:5 (25.1 min) to 95:5 (30 min)

Method C4:

detection at 210-225 nm, optionally coupled to a mass analyser Waters LCT Premier, electrospray positive ion mode
column: Waters ACQUITY UPLC® BEH™ C18 1.7 μm (150×2.1 mm) at 50° C.
solvent: H₂O+1% FA: ACN+1% FA (flow 1 ml/min)
gradient: 95:5 (0 min) to 95:5 (1.80 min) to 80:20 (1.85 min) to 80:20 (3 min) to 60:40 (23 min) to 2:98 (23.1 min) to 2:98 (25 min) to 95:5 (25.1 min) to 95:5 (30 min)

Method D: detection at 214 nm
column: Waters X-Bridge C18 3.5 μm 2.1×150 mm
solvent: H₂O+0.5% TFA: ACN (flow 0.55 ml/min)
gradient: 90:10 (0 min) to 40:60 (5 min) to 1:99 (15 min)

Method E: detection at 210-225 nm, optionally coupled to a mass analyser Waters LCT Premier, electrospray positive ion mode
column: Waters ACQUITY UPLC® BEH™ C18 1.7 μm (150×2.1 mm) at 50° C.
solvent: H₂O+1% FA: ACN+1% FA (flow 0.9 ml/min)
gradient: 95:5 (0 min) to 95:5 (2 min) to 35:65 (3 min) to 65:35 (23.5 min) to 5:95 (24 min) to 95:5 (26 min) to 95:5 (30 min)

General Preparative HPLC Purification Procedure:

The crude peptides were purified either on an Äkta Purifier System or on a Jasco semiprep HPLC System. Preparative RP-C18-HPLC columns of different sizes and with different flow rates were used depending on the amount of crude peptide to be purified. Acetonitrile+0.05 to 0.1% TFA (B) and water+0.05 to 0.1% TFA (A) were employed as eluents. Alternatively, a buffer system consisting of acetonitrile and water with minor amounts of acetic acid was used. Product-containing fractions were collected and lyophilized to obtain the purified product, typically as TFA or acetate salt.

Solubility and Stability-Testing of Exendin-4 Derivatives

Prior to the testing of solubility and stability of a peptide batch, its content was determined Therefore, two parameters were investigated, its purity (HPLC-UV) and the amount of salt load of the batch (ion chromatography).

For solubility testing, the target concentration was 1.0 mg/mL pure compound. Therefore, solutions from solid samples were prepared in different buffer systems with a concentration of 1.0 mg/mL compound based on the previously determined content. HPLC-UV was performed after 2 h of gentle agitation from the supernatant, which was obtained by 20 min of centrifugation at 4000 rpm.

The solubility was then determined by comparison with the UV peak areas obtained with a stock solution of the peptide at a concentration of 2 mg/mL in pure water or a variable amount of acetonitrile (optical control that all of the compound was dissolved). This analysis also served as starting point (t0) for the stability testing.

For stability testing, an aliquot of the supernatant obtained for solubility was stored for 7 days at 25° C. or 40° C. After that time course, the sample was centrifuged for 20 min at 4000 rpm and the supernatant was analysed with HPLC-UV.

For determination of the amount of the remaining peptide, the peak areas of the target compound at t0 and t7 were compared, resulting in “% remaining peptide”, following the equation

% remaining peptide=[(peak area peptide t7)×100]/peak area peptide t0.

The amount of soluble degradation products was calculated from the comparison of the sum of the peak areas from all observed impurities reduced by the sum of peak areas observed at t0 (i.e. to determine the amount of newly formed peptide-related species). This value was given in percentual relation to the initial amount of peptide at t0, following the equation:

% soluble degradation products={[(peak area sum of impurities t7)−(peak area sum of impurities t0)]×100}/peak area peptide t0

The potential difference from the sum of “% remaining peptide” and “% soluble degradation products” to 100% reflects the amount of peptide which did not remain soluble upon stress conditions following the equation

% precipitate=100−([% remaining peptide]+[% soluble degradation products])

This precipitate includes non-soluble degradation products, polymers and/or fibrils, which have been removed from analysis by centrifugation.

The chemical stability is expressed as “% remaining peptide”.

Anion Chromatography

Instrument: Dionex ICS-2000, pre/column: Ion Pac AG-18 2×50 mm (Dionex)/AS18 2×250 mm (Dionex), eluent: aqueous sodium hydroxide, flow: 0.38 mL/min, gradient: 0-6 min: 22 mM KOH, 6-12 min: 22-28 mM KOH, 12-15 min: 28-50 mM KOH, 15-20 min: 22 mM KOH, suppressor: ASRS 300 2 mm, detection: conductivity.

As HPLC/UPLC method, method D or E has been used.

In Vitro Cellular Assays for GLP-1 Receptor, Glucagon Receptor and GIP Receptor Efficacy

Agonism of compounds for the receptors was determined by functional assays measuring cAMP response of HEK-293 cell lines stably expressing human GIP, GLP-1 or glucagon receptor.

cAMP content of cells was determined using a kit from Cisbio Corp. (cat. no. 62AM4PEC) based on HTRF (Homogenous Time Resolved Fluorescence). For preparation, cells were split into T175 culture flasks and grown overnight to near confluency in medium (DMEM/10% FBS). Medium was then removed and cells washed with PBS lacking calcium and magnesium, followed by proteinase treatment with accutase (Sigma-Aldrich cat. no. A6964). Detached cells were washed and resuspended in assay buffer (1×HBSS; 20 mM HEPES, 0.1% BSA, 2 mM IBMX) and cellular density determined They were then diluted to 400000 cells/ml and 25 μl-aliquots dispensed into the wells of 96-well plates. For measurement, 25 μl of test compound in assay buffer was added to the wells, followed by incubation for 30 minutes at room temperature. After addition of HTRF reagents diluted in lysis buffer (kit components), the plates were incubated for 1 hr, followed by measurement of the fluorescence ratio at 665/620 nm. In vitro potency of agonists was quantified by determining the concentrations that caused 50% activation of maximal response (EC50).

Glucose Lowering in Female Diabetic Dbdb-Mice

Female diabetic dbdb-mice (BKS.Cg-+Leprdb/+Leprdb/OlaHsd) 10 weeks of age at study start were used. Mice were habituated to feeding and housing conditions for at least 2 weeks. 7 days prior to study start, HbA1c were determined to allocate mice to groups, aiming to spread low, medium and high HbA1c-values and in consequence the group-means (n=8), as equally as possible. On the day of study, food was removed, directly before sampling for baseline glucose assessment (t=0 min) Immediately afterwards, compounds or vehicle (phosphor buffered saline, PBS) were administered subcutaneously, 100 μg/kg, 10 ml/kg. Afterwards, blood samples were drawn by tail tip incision at 15, 30, 60, 90, 120, 150, 180, 240, 360, 480 min and 24 h. Food was re-offered after the 480 min-sampling.

Data were analysed by 2-W-ANOVA on repeated measurements, followed by Dunnett's test as post-hoc assessment, level of significance p<0.05.

### EXAMPLES

The invention is further illustrated by the following examples.

### Example 1

### Synthesis of SEQ ID NO: 6

The solid phase synthesis was carried out on Novabiochem Rink-Amide resin (4-(2′,4′-Dimethoxyphenyl-Fmoc-aminomethyl)-phenoxyacetamido-norleucylaminomethyl resin), 100-200 mesh, loading of 0.23 mmol/g. The Fmoc-synthesis strategy was applied with HBTU/DIPEA-activation. The peptide was cleaved from the resin with King's cocktail (D. S. King, C. G. Fields, G. B. Fields, Int. J. Peptide Protein Res. 36, 1990, 255-266). The crude product was purified via preparative HPLC on a Waters column (XBridge, BEH130, Prep C18 5 μM) using an acetonitrile/water gradient (both buffers with 0.1% TFA). Finally, the molecular mass of the purified peptide was confirmed by LC-MS.

### Example 2

### Synthesis of SEQ ID NO: 8

The solid phase synthesis was carried out on Novabiochem Rink-Amide resin (4-(2′,4′-Dimethoxyphenyl-Fmoc-aminomethyl)-phenoxyacetamido-norleucylaminomethyl resin), 100-200 mesh, loading of 0.34 mmol/g. The Fmoc-synthesis strategy was applied with HBTU/DIPEA-activation. The peptide was cleaved from the resin with King's cocktail (D. S. King, C. G. Fields, G. B. Fields, Int. J. Peptide Protein Res. 36, 1990, 255-266). The crude product was purified via preparative HPLC on a Waters column (XBridge, BEH130, Prep C18 5 μM) using an acetonitrile/water gradient (both buffers with 0.1% TFA). Finally, the molecular mass of the purified peptide was confirmed by LC-MS.

In an analogous way, the other peptides listed in Table 2 were synthesized.

TABLE 2
list of synthesized peptides and comparison
of calculated vs. found molecular weight

| SEQ ID NO | calc. Mass | found mass |
|---|---|---|
| 5 | 4169.6 | 4169.1 |
| 6 | 4180.9 | 4181.6 |
| 7 | 4155.6 | 4156.2 |
| 8 | 4169.5 | 4168.4 |
| 9 | 4167.6 | 4166.8 |
| 10 | 4162.6 | 4163.0 |
| 11 | 4153.6 | 4153.7 |
| 12 | 4169.6 | 4169.8 |
| 13 | 4153.6 | 4152.4 |
| 14 | 4167.6 | 4166.4 |
| 15 | 4162.6 | 4163.2 |
| 16 | 4167.6 | 4168.0 |
| 17 | 4165.7 | 4165.9 |
| 18 | 4169.5 | 4168.7 |
| 19 | 4176.6 | 4176.9 |
| 20 | 4181.6 | 4181.4 |
| 21 | 4176.6 | 4176.9 |
| 22 | 4167.6 | 4167.6 |
| 23 | 4183.6 | 4182.9 |
| 26 | 4164.6 | 4163.9 |
| 27 | 4127.5 | 4126.8 |

In an analogous way, the following peptides of Table 3 can be synthesized:

TABLE 3
List of peptides that can be synthesized
in an analogous way.
SEQ ID NO
24
25

### Example 3

### Chemical Stability and Solubility

Solubility and chemical stability of peptidic compounds were assessed as described in Methods. The results are given in Table 4.

TABLE 4
Chemical stability and solubility

| | | | | solubility | |
|---|---|---|---|---|---|
| SEQ | Stability | | | [mg/ml] | |
| ID NO | pH 4 .5 | pH 7.4 | Temperature | pH 4.5 | pH 7.4 |
| 1 | 100 | 77.5 | 25° C. | 933.6 | >1000 |
| 6 | 92.0 | 94.4 | 40° C. | 911.1 | 966.7 |
| 10 | 98.8 | 97.0 | 40° C. | 950.0 | 710.0 |
| 11 | 94.0 | 98.1 | 40° C. | 922.9 | 969.4 |
| 12 | 94.1 | 95.7 | 40° C. | 992.2 | 971.6 |

### Example 4

### In Vitro Data on GLP-1 and Glucagon Receptor

Potencies of peptidic compounds at the GLP-1 and glucagon receptors were determined by exposing cells expressing human glucagon receptor (hGlucagon R) or human GLP-1 receptor (hGLP-1 R) to the listed compounds at increasing concentrations and measuring the formed cAMP as described in Methods.

The results are shown in Table 5:

TABLE 5
EC50 values of exendin-4 derivatives at
GLP-1 and Glucagon receptors
(indicated in pM)

| | EC50 | EC50 |
|---|---|---|
| SEQ ID NO | hGLP-1R | hGlucagon-R |
| 5 | 1.7 | 6.2 |
| 6 | 2.7 | 5.0 |
| 7 | 4.9 | 24.3 |
| 8 | 3.1 | 15.9 |
| 9 | 8.5 | 20.6 |
| 10 | 1.2 | 5.3 |
| 11 | 1.5 | 35.6 |
| 12 | 1.6 | 47.5 |
| 13 | 0.9 | 68.0 |
| 14 | 1.0 | 60.3 |
| 15 | 1.0 | 35.6 |
| 16 | 1.4 | 44.9 |
| 17 | 1.6 | 77.5 |
| 18 | 1.0 | 18.3 |
| 19 | 3.7 | 8.2 |
| 20 | 6.0 | 10.1 |
| 21 | 2.6 | 7.9 |
| 22 | 1.7 | 4.7 |
| 23 | 0.8 | 26.7 |
| 26 | 1.9 | 8.9 |
| 27 | 4.4 | 26.1 |

### Example 5

### Glucose Lowering in Female Diabetic dbdb-Mice

Female db/db-mice, received 100 μg/kg of SEQ ID NO: 10 or phosphate buffered saline (vehicle control) subcutaneously, at time 0 min. SEQ ID NO: 10 immediately lowered glucose values (baseline on average at 28 mmol/l), reaching the maximal effect of ˜12 mmol/1 glucose reduction.

SEQ ID NO: 10 reached a statistical significant reduction of glucose compared to vehicle control from t=60 min until 240 min (p<0.05, 2-way-ANOVA on repeated measures, followed by Dunnett's post-hoc test).

TABLE 6
Sequences

| SEQ | |
|---|---|
| ID | Sequence |
| 1 | H-G-E-G-T-F-T-S-D-L-S-K-Q-M-E-E-E-A-V-R-L-F-I-E-W-L-K-N-G-G-P-S-S- |
| | G-A-P-P-P-S-NH2 |
| 2 | H-A-E-G-T-F-T-S-D-V-S-S-Y-L-E-G-Q-A-A-K-E-F-I-A-W-L-V-K-G-R-NH2 |
| 3 | H-S-Q-G-T-F-T-S-D-Y-S-K-Y-L-D-S-R-R-A-Q-D-F-V-Q-W-L-M-N-T |
| 4 | H-A-E-G-T-F-T-S-D-V-S-S-Y-L-E-G-Q-A-A-K((S)-4-Carboxy-4- |
| | hexadecanoylamino-butyryl-)-E-F-I-A-W-L-V-R-G-R-G |
| 5 | H-S-Q-G-T-F-T-S-D-L-S-K-Q-L-D-E-Q-R-A-K-E-F-I-E-W-L-I-A-G-G-P-S-S-G- |
| | A-P-P-P-S-NH2 |
| 6 | H-S-Q-G-T-F-T-S-D-L-S-K-Q-L-D-E-Q-R-A-K-E-F-I-E-W-L-I-A-G-G-P-V-S- |
| | G-A-P-P-P-S-NH2 |
| 7 | H-dSer-Q-G-T-F-T-S-D-L-S-K-Q-L-D-E-Q-R-A-K-D-F-I-E-W-L-I-A-G-G-P-S- |
| | S-G-A-P-P-P-S-NH2 |
| 8 | H-dSer-Q-G-T-F-T-S-D-L-S-K-Q-L-D-E-Q-R-A-(S)MeLys-D-F-I-E-W-L-I-A-G- |
| | G-P-S-S-G-A-P-P-P-S-NH2 |
| 9 | H-dSer-Q-G-T-F-T-S-D-L-S-K-Q-L-D-E-Q-R-A-K-D-F-I-E-W-L-I-A-G-G-P-V- |
| | S-G-A-P-P-P-S-NH2 |
| 10 | H-Aib-H-G-T-F-T-S-D-L-S-K-Q-L-D-E-Q-R-A-K-D-F-I-E-W-L-I-A-G-G-P-S-S- |
| | G-A-P-P-P-S-NH2 |
| 11 | H-Aib-Q-G-T-F-T-S-D-L-S-K-Q-L-D-E-Q-R-A-Q-D-F-I-E-W-L-I-A-G-G-P-S-S- |
| | G-A-P-P-P-S-NH2 |
| 12 | H-Aib-Q-G-T-F-T-S-D-L-S-K-Q-L-D-E-Q-R-A-K-D-F-I-E-W-L-I-S-G-G-P-S-S- |
| | G-A-P-P-P-S-NH2 |
| 13 | H-Aib-Q-G-T-F-T-S-D-L-S-K-Q-L-D-E-Q-R-A-K-D-F-I-E-W-L-I-A-G-G-P-S-S- |
| | G-A-P-P-P-S-NH2 |
| 14 | H-Aib-Q-G-T-F-T-S-D-L-S-K-Q-L-D-E-Q-R-A-(S)MeLys-D-F-I-E-W-L-I-A-G- |
| | G-P-S-S-G-A-P-P-P-S-NH2 |
| 15 | H-Aib-Q-G-T-F-T-S-D-L-S-K-Q-L-D-E-Q-R-A-H-D-F-I-E-W-L-I-A-G-G-P-S-S- |
| | G-A-P-P-P-S-NH2 |
| 16 | H-Aib-Q-G-T-F-T-S-D-L-S-K-Q-L-D-E-Q-R-A-K-E-F-I-E-W-L-I-A-G-G-P-S-S- |
| | G-A-P-P-P-S-NH2 |
| 17 | H-Aib-Q-G-T-F-T-S-D-L-S-K-Q-L-D-E-Q-R-A-K-D-F-I-E-W-L-I-A-G-G-P-V- |
| | S-G-A-P-P-P-S-NH2 |
| 18 | H-Aib-Q-G-T-F-T-S-D-L-S-K-Q-L-D-E-Q-R-A-Q-D-F-I-E-W-L-I-S-G-G-P-S-S- |
| | G-A-P-P-P-S-NH2 |
| 19 | H-S-H-G-T-F-T-S-D-L-S-K-Q-L-D-E-Q-R-A-K-D-F-I-E-W-L-I-A-G-G-P-V-S- |
| | G-A-P-P-P-S-NH2 |
| 20 | H-dSer-Q-G-T-F-T-S-D-L-S-K-Q-L-D-E-Q-R-A-K-E-F-I-E-W-L-I-A-G-G-P-V- |
| | S-G-A-P-P-P-S-NH2 |
| 21 | H-dSer-H-G-T-F-T-S-D-L-S-K-Q-L-D-E-Q-R-A-K-D-F-I-E-W-L-I-A-G-G-P-V- |
| | S-G-A-P-P-P-S-NH2 |
| 22 | H-S-Q-G-T-F-T-S-D-L-S-K-Q-L-D-E-Q-R-A-K-D-F-I-E-W-L-I-A-G-G-P-V-S- |
| | G-A-P-P-P-S-NH2 |
| 23 | H-Aib-Q-G-T-F-T-S-D-L-S-K-Q-L-D-E-Q-R-A-K-E-F-I-E-W-L-I-S-G-G-P-S-S- |
| | G-A-P-P-P-S-NH2 |
| 24 | H-S-Q-G-T-F-T-S-D-L-S-K-Q-L-D-E-Q-R-A-(S)MeLys-D-F-I-E-W-L-I-A-G-G- |
| | P-S-S-G-A-P-P-P-S-NH2 |
| 25 | H-dSer-Q-G-T-F-T-S-D-L-S-K-Q-L-D-E-Q-R-A-(S)MeLys-D-F-I-E-W-L-I-A-G- |
| | G-P-V-S-G-A-P-P-P-S-NH2 |
| 26 | H-dSer-H-G-T-F-T-S-D-L-S-K-Q-L-D-E-Q-R-A-K-D-F-I-E-W-L-I-A-G-G-P-S- |
| | S-G-A-P-P-P-S-NH2 |
| 27 | H-dSer-Q-G-T-F-T-S-D-L-S-K-Q-L-D-E-Q-K-A-K-D-F-I-E-W-L-I-A-G-G-P-S- |
| | S-G-A-P-P-P-S-NH2 |

1. A peptidic compound having the formula (I):
R¹—Z—R² (I)
wherein Z is a peptide moiety having the formula (II)
(II)
His-X2-X3-Gly-Thr-Phe-Thr-Ser-Asp-Leu-Ser-Lys-Gln-
Leu-Asp-Glu-Gln-X18-Ala-X20-X21-Phe-Ile-Glu-Trp-
Leu-Ile-X28-Gly-Gly-Pro-X32-Ser-Gly-Ala-Pro-Pro-
Pro-Ser
X2 represents an amino acid residue selected from Ser, D-Ser, or Aib,
X3 represents an amino acid residue selected from Gln or His,
X18 represents an amino acid residue selected from Arg or Lys
X20 represents an amino acid residue selected from Lys, Gln, His or (S)MeLys,
X21 represents an amino acid residue selected from Asp or Glu,
X28 represents an amino acid residue selected from Ser or Ala,
X32 represents an amino acid residue selected from Ser or Val,
R¹ represents NH₂, and
R² represents OH or NH₂,
or a salt or solvate thereof.

2. The compound of claim 1,
which is a GLP1 and Glucagon receptor agonist.

3. The compound according to any one of claim 1, wherein R² is NH₂.

4. The compound of claim 1, wherein the peptidic compound has a relative activity of at least 0.1% compared to that of natural glucagon at the glucagon receptor.

5. The compound of claim 1, wherein the peptidic compound exhibits a relative activity of at least 0.1% compared to that of GLP-1(7-36) at the GLP-1 receptor.

6. The compound of claim 1, wherein
X2 represents an amino acid residue selected from Ser, D-Ser or Aib,
X3 represents an amino acid residue selected from Gln or His,
X18 represents Arg
X20 represents an amino acid residue selected from Lys, Gln, His, or (S)MeLys,
X21 represents an amino acid residue selected from Asp or Glu,
X28 represents an amino acid residue selected from Ser or Ala, and
X32 represents an amino acid residue selected from Ser or Val.

7. The compound of claim 1, wherein
X2 represents an amino acid residue selected from Ser, D-Ser or Aib,
X3 represents an amino acid residue selected from Gln or His,
X18 represents an amino acid residue selected from Arg or Lys,
X20 represents Lys,
X21 represents an amino acid residue selected from Asp or Glu,
X28 represents an amino acid residue selected from Ser or Ala, and
X32 represents an amino acid residue selected from Ser or Val.

8. The compound of claim 1, wherein
X2 represents an amino acid residue selected from Ser, D-Ser or Aib,
X3 represents Gln,
X18 represents Arg,
X20 represents (S)MeLys,
X21 represents Asp,
X28 represents Ala, and
X32 represents an amino acid residue selected from Ser or Val.

9. The compound of claim 1, wherein
X2 represents Aib,
X3 represents Gln,
X18 represents Arg,
X20 represents an amino acid residue selected from Lys or Gln,
X21 represents an amino acid residue selected from Asp or Glu,
X28 represents Ser, and
X32 represents Ser.

10. The compound of claim 1, wherein
X2 represents an amino acid residue selected from Ser, D-Ser or Aib,
X3 represents an amino acid residue selected from Gln or His,
X18 represents Arg,
X20 represents an amino acid residue selected from Lys or (S)MeLys,
X21 represents an amino acid residue selected from Asp or Glu,
X28 represents Ala, and
X32 represents Val.

11. The compound of claim 1, wherein
X2 represents an amino acid residue selected from Ser or Aib,
X3 represents an amino acid residue selected from Gln or His,
X18 represents Arg,
X20 represents an amino acid residue selected from Lys or Gln,
X21 represents an amino acid residue selected from Asp or Glu,
X28 represents an amino acid residue selected from Ser or Ala, and
X32 represents an amino acid residue selected from Ser or Val.

12. The compound of claim 1, wherein the compound is any one of SEQ ID NO: 5-27, as well as a salt or solvate thereof.

13. A pharmaceutical composition comprising the compound of claim 1.

14. The pharmaceutical composition of claim 13 together with at least one pharmaceutically acceptable carrier.

15. The compound for use according to pharmaceutical composition of claim 13 together with at least one additional therapeutically active agent, wherein the additional therapeutically active agent is selected from the group consisting of insulin and insulin derivatives; GLP-1; GLP-1 analogues; GLP-1 receptor agonists; polymer bound GLP-1 and GLP-1 analogues; dual GLP1/GIP agonists; PYY3-36; pancreatic polypeptide; glucagon receptor agonists; GIP receptor agonists or antagonists; ghrelin antagonists or inverse agonists; xenin; DDP-IV inhibitors; SGLT2 inhibitors; dual SGLT2/SGLT1 inhibitors; biguanides; thiazolidinediones; dual PPAR agonists; sulfonylureas; meglitinides; alpha-glucosidase inhibitors; amylin and pramlintide; GPR119 agonists; GPR40 agonists; GPR120 agonists; GPR142 agonists; systemic or low-absorbable TGR5 agonists; cycloset; inhibitors of 11-beta-HSD; activators of glucokinase inhibitors of DGAT; inhibitors of protein tyrosinephosphatase 1; inhibitors of glucose-6-phosphatase; inhibitors of fructose-1,6-bisphosphatase; inhibitors of glycogen phosphorylase; inhibitors of phosphoenol pyruvate carboxykinase; inhibitors of glycogen synthase kinase; inhibitors of pyruvate dehydrogenase kinase; alpha2-antagonists; CCR-2 antagonists; modulators of glucose transporter-4; somatostatin receptor 3 agonists; HMG-CoA-reductase inhibitors; fibrates; nicotinic acid and derivatives thereof; nicotinic acid receptor 1 agonists; PPAR-alpha, gamma, or alpha/gamma agonists or modulators; PPAR-delta agonists; ACAT inhibitors; cholesterol absorption inhibitors; bile acid-binding substances; IBAT inhibitors; MTP inhibitors; modulators of PCSK9; LDL receptor up-regulators by liver selective thyroid hormone receptor β agonists; HDL-raising compounds; lipid metabolism modulators; PLA2 inhibitors; ApoA-I enhancers; thyroid hormone receptor agonists; cholesterol synthesis inhibitors; omega-3 fatty acids and derivatives thereof; substances for the treatment of obesity selected from the group consisting of sibutramine, tesofensine, tetrahydrolipstatin, CB-1 receptor antagonists, MCH-1 antagonists, MC4 receptor agonists and partial agonists, NPY5 or NPY2 antagonists, NPY4 agonists, beta-3-agonists, leptin or leptin mimetics, agonists of the 5HT2c receptor, combinations of bupropione/naltrexone, combinations of bupropione/zonisamide, combinations of bupropione/phentermine, combinations of pramlintide/metreleptin, + and combinations of phentermine/topiramate; lipase inhibitors; angiogenesis inhibitors; H3 antagonists; AgRP inhibitors; triple monoamine uptake inhibitors; MetAP2 inhibitors; nasal formulation of the calcium channel blocker diltiazem; inhibitors of fibroblast growth factor receptor 4; prohibitin targeting peptide-1; and drugs for influencing high blood pressure, chronic heart failure, or atherosclerosis selected from the group consisting of angiotensin II receptor antagonists, ACE inhibitors, ECE inhibitors, diuretics, beta-blockers, calcium antagonists, centrally acting hypertensives, antagonists of the alpha-2-adrenergic receptor, inhibitors of neutral endopeptidase, and thrombocyte aggregation inhibitors.

16. A method of treating, preventing, or delaying the progression of a disease or disorder comprising administering to a patient in need thereof the pharmaceutical composition of claim 13 hyperglycemia, type 2 diabetes, impaired glucose tolerance, type 1 diabetes, obesity, metabolic syndrome, neurodegenerative disorders, bulimia, binge eating, atherosclerosis, hypertension, IGT, dyslipidemia, coronary heart disease, hepatic steatosis, and beta-blocker poisoning.

17. The method of claim 15, wherein the disease or disorder is selected from the group consisting of hyperglycemia, type 2 diabetes, obesity.

18. (canceled)

19. A method of treating hyperglycemia, type 2 diabetes, or obesity in a patient, the method comprising administering to the patient an effective amount of at least one compound of formula I according to claim 1 and an effective amount of at least one additional compound for treating hyperglycemia, type 2 diabetes, or obesity.

20. The method of claim 19 wherein the effective amount of at least one at least one compound of formula I and the additional compound are administered to the patient simultaneously.

21. The method of claim 19 wherein the effective amount of at least one compound of formula I and the additional compound are administered to the patient sequentially.

22. The method of claim 16, wherein the method delays the progression of impaired glucose tolerance (IGT) to type 2 diabetes or type 2 diabetes to insulin-requiring diabetes.

23. The method of claim 16, wherein the method regulates appetite, induces satiety, prevents weight regain after successful weight loss, or inhibits the motility of the gastro-intestinal tract.

24. A method of imaging the gastro-intestinal tract, wherein the method comprises at least one at least one compound of formula I according to claim 1 and a technique selected from the group consisting of X-ray, CT-scanning, and NMR-scanning.


SEQUENCE LISTING (27 sequences)

SEQ ID NO: 1 - protein, 39 aa, Heloderma suspectum
  MOD_RES (39)..(39): Amidated C-terminus
HGEGTFTSDLSKQMEEEAVRLFIEWLKNGGPSSGAPPPS

SEQ ID NO: 2 - protein, 30 aa, Homo sapiens
  MOD_RES (30)..(30): Amidated C-terminus
HAEGTFTSDVSSYLEGQAAKEFIAWLVKGR

SEQ ID NO: 3 - protein, 29 aa, Homo sapiens
HSQGTFTSDYSKYLDSRRAQDFVQWLMNT

SEQ ID NO: 4 - protein, 31 aa, Artificial sequence
  Liraglutide
HAEGTFTSDVSSYLEGQAAKEFIAWLVRGRG

SEQ ID NO: 5 - protein, 39 aa, Artificial Sequence
  Exendin-4 analogue
HSQGTFTSDLSKQLDEQRAKEFIEWLIAGGPSSGAPPPS

SEQ ID NO: 6 - protein, 39 aa, Artificial Sequence
  Exendin-4 analogue
HSQGTFTSDLSKQLDEQRAKEFIEWLIAGGPVSGAPPPS

SEQ ID NO: 7 - protein, 39 aa, Artificial Sequence
  Exendin-4 analogue
HXQGTFTSDLSKQLDEQRAKDFIEWLIAGGPSSGAPPPS

SEQ ID NO: 8 - protein, 39 aa, Artificial Sequence; identical to SEQ ID NO: 7
  Exendin-4 analogue

SEQ ID NO: 9 - protein, 39 aa, Artificial Sequence
  Exendin-4 analogue
HXQGTFTSDLSKQLDEQRAKDFIEWLIAGGPVSGAPPPS

SEQ ID NO: 10 - protein, 39 aa, Artificial Sequence
  Exendin-4 analogue
HXHGTFTSDLSKQLDEQRAKDFIEWLIAGGPSSGAPPPS

SEQ ID NO: 11 - protein, 39 aa, Artificial Sequence
  Exendin-4 analogue
HXQGTFTSDLSKQLDEQRAQDFIEWLIAGGPSSGAPPPS

SEQ ID NO: 12 - protein, 39 aa, Artificial Sequence
  Exendin-4 analogue
HXQGTFTSDLSKQLDEQRAKDFIEWLISGGPSSGAPPPS

SEQ ID NO: 13 - protein, 39 aa, Artificial Sequence; identical to SEQ ID NO: 7
  Exendin-4 analogue

SEQ ID NO: 14 - protein, 39 aa, Artificial Sequence; identical to SEQ ID NO: 7
  Exendin-4 analogue

SEQ ID NO: 15 - protein, 39 aa, Artificial Sequence
  Exendin-4 analogue
HXQGTFTSDLSKQLDEQRAHDFIEWLIAGGPSSGAPPPS

SEQ ID NO: 16 - protein, 39 aa, Artificial Sequence
  Exendin-4 analogue
HXQGTFTSDLSKQLDEQRAKEFIEWLIAGGPSSGAPPPS

SEQ ID NO: 17 - protein, 39 aa, Artificial Sequence; identical to SEQ ID NO: 9
  Exendin-4 analogue

SEQ ID NO: 18 - protein, 39 aa, Artificial Sequence
  Exendin-4 analogue
HXQGTFTSDLSKQLDEQRAQDFIEWLISGGPSSGAPPPS

SEQ ID NO: 19 - protein, 39 aa, Artificial Sequence
  Exendin-4 analogue
HSHGTFTSDLSKQLDEQRAKDFIEWLIAGGPVSGAPPPS

SEQ ID NO: 20 - protein, 39 aa, Artificial Sequence
  Exendin-4 analogue
HXQGTFTSDLSKQLDEQRAKEFIEWLIAGGPVSGAPPPS

SEQ ID NO: 21 - protein, 39 aa, Artificial Sequence
  Exendin-4 analogue
HXHGTFTSDLSKQLDEQRAKDFIEWLIAGGPVSGAPPPS

SEQ ID NO: 22 - protein, 39 aa, Artificial Sequence
  Exendin-4 analogue
HSQGTFTSDLSKQLDEQRAKDFIEWLIAGGPVSGAPPPS

SEQ ID NO: 23 - protein, 39 aa, Artificial Sequence
  Exendin-4 analogue
HXQGTFTSDLSKQLDEQRAKEFIEWLISGGPSSGAPPPS

SEQ ID NO: 24 - protein, 39 aa, Artificial Sequence
  Exendin-4 analogue
HSQGTFTSDLSKQLDEQRAKDFIEWLIAGGPSSGAPPPS

SEQ ID NO: 25 - protein, 39 aa, Artificial Sequence; identical to SEQ ID NO: 9
  Exendin-4 analogue

SEQ ID NO: 26 - protein, 39 aa, Artificial Sequence; identical to SEQ ID NO: 10
  Exendin-4 analogue

SEQ ID NO: 27 - protein, 39 aa, Artificial Sequence
  Exendin-4 analogue
HXQGTFTSDLSKQLDEQKAKDFIEWLIAGGPSSGAPPPS